(19) United States
(12) Patent Application Publication
(10) Pub. No.: US 2012/0143021 A1
(43) Pub. Date: Jun. 7, 2012

(54) DEVICE, SYSTEM AND METHOD FOR MEAL DETECTION FOR INTERFACING WITH AND CONTROLLING AUXILIARY DEVICES ASSOCIATED THEREWITH

(76) Inventor: Ron Nagar, Tel Aviv (IL)

(21) Appl. No.: 13/388,365

(22) PCT Filed: Aug. 2, 2010
(86) PCT No.: PCT/IL2010/000623

§ 371 Date: Feb. 1, 2012

Related U.S. Application Data
(60) Provisional application No. 61/230,734, filed on Aug. 2, 2009.

(51) Int. Cl. A61M 5/168; A61F 2/04; A61B 5/145; A61B 5/103; A61B 7/00

(52) U.S. Cl. 600/301; 604/66; 600/37; 606/191; 604/8

(57) ABSTRACT

The present invention relates to a device, system and method for detecting meal event in a subject and in particular to such a device, system and method for communicating and controlling auxiliary devices and/or systems associated therewith.

### FIELD OF THE INVENTION

The present invention relates to a device, system and method for detecting meal event in a subject and in particular to such a device, system and method for communicating and controlling auxiliary devices and/or systems associated therewith.

### BACKGROUND OF THE INVENTION

Many of our daily activities revolve about timing of a meal. For example many medicaments must be taken in relation to a meal either before, with or after the meal. Although some medicament administration is associated with an acute condition that requires a short course of drug treatment, for example over a week, however there are many chronic medical conditions that require a patient to check or administer a drug relative to the timing of each meal on a daily basis, amounting to about two to three times a day. In some chronic conditions the administration of a drug, treatment or medicament is required over an extended period of time, on the order of months if not years where each administration of the drug may be relative to the timing of a meal. Such a long term requirement is difficult to maintain at all times. Therefore, there are many occasions when patients of chronic diseases omits to take a drug that should have be taken or administered relative to the timing of the meal, before, after or during a meal. One such chronic illness is diabetes.

Diabetes is a very serious illness affecting millions of people today. Many diabetic patients require injection of insulin to maintain proper blood glucose levels which is an essential requirement for their survival. Many of the insulin dependent patients need to take insulin prior to meals in order to control their post meal blood glucose level. Injections of insulin may be performed in a number of modalities, one of which uses a drug delivery systems that are usually controlled by the user. There are many occasions when diabetic patients have meals without taking insulin. This result in relatively high post meal blood glucose levels, and over years, contributes to complications.

There are several methods and systems described in the literature that provide a signal when the patient is eating. Some of the systems are non invasive while other meal detection systems are invasive and/or implanted. Such meal detection system provide for modifying patient behavior for example to limit food intake to prevent obesity. An example of such meal detection systems are described for example U.S. Pat. No. 4,823,808 to Clegg, U.S. Pat. No. 6,135,950 to Adams, US Publication No. 2006/0064037 to Shalon, PCT Publication No. WO2006/033104 to Shalon et al U.S. Pat. No. 5,263,491 to Thornton, WO2008/149341 to Shalon et al, and papers by Zhang et al, “Detection of Activities by Wireless Sensors Surveillance: Eating and Drinking” Sensors 2009, 9, 1499-1517; which describe non-invasive systems that provide meal detection signal.

Other solution provide an invasive and/or implanted systems for detecting a meal for example U.S. Pat. No. 7,502,649 to BEN HAIM, PCT Publication No. WO2004/112563 to BEN HAIM, and PCT Publication No. WO2008/070575 to Soffer et al.

However despite varying levels of invasiveness the meal detection systems are complex and often suffer from high rate of false detection and/or missed detection. Missed or false detection are not tolerable in systems that are designed to provide medication based on the detection of event.

### SUMMARY OF THE INVENTION

Meal detection becomes an important indicator that may affect the way in which acute or chronic conditions are managed. The disease and drug administration management greatly dependent on the nature of the condition, some such chronic conditions management directly relate to the recognition of a meal, for example obesity to control the caloric intake, diabetes to control blood glucose levels, atherosclerosis to control blood cholesterol and fats. Therefore meal detection may therefore become an integral part of disease management wherein the condition being treated is directly or indirectly related to a meal event and/or caloric intake during such an event.

For example, diabetic patients require close and accurate control of their blood glucose level in an attempt reach a blood glucose balance or homeostasis by the controllable administration of insulin with respect to monitored blood glucose levels. For diabetic patients, there are some new insulin delivery systems which infuse insulin drug subcutaneously. Some of these systems have automatic insulin infusion capability that is based on glucose reading. These systems usually include at least a glucose sensor and an insulin infusion pump. An algorithm is embedded in such systems that are used to calculate and adjust the amount of infused insulin based, among other parameters, on the glucose level input. These systems may require user acknowledgement before insulin infusion and hereinafter will be referred to as—Semi Closed Loop Insulin Delivery Systems” (SCLIDS), or infuse insulin without user intervention, which will hereinafter be referred to as “Fully Closed Loop Insulin Delivery Systems” (FCLIDS). The SCLIDS or FCLIDS need to respond fast to changes in blood glucose levels since the insulin delivered by them is infused subcutaneously and it takes relatively long time to reach the blood circulation where it can affect the patient's blood glucose level.

For this purposes SCLIDS or FCLIDS systems may use knowledge of a meal event to better control and manage the blood glucose fluctuations in trying to reach blood glucose equilibrium or homeostasis. Some SCLIDS or FCLIDS systems infer a meal events based on several internal glucose readings. A better way is to have other methods that can detect meals since it take time from start of meal until glucose level changes, time that can be used for the insulin to diffuse to the blood, had the insulin been infused immediately with the meal.

In SCLIDS the user can manually indicate or input the onset of a meal event to the system, however this is generally unreliable as many meal events may be missed due to user forgetfulness. Accordingly it is desirable to use less user interventions in systems designed to treat chronic conditions, since over time, users makes mistakes or forget to inform the system of such an event.

There is an unmet need for, and it would be highly useful to have, a system and a method for integrating a drug delivery device and a meal detection device that improve management of medical conditions over time, and in particular to system where the medical condition is chronic, for example such as diabetes, atherosclerosis or obesity.

For this purposes SCLIDS or FCLIDS, or the like drug delivery systems may use knowledge of meal event to trigger initial drug infusion to face expected post meal glucose increase. Some SCLIDS or FCLIDS systems decide on meal events based on several glucose readings. A better way is to have other methods that can detect meals since it take time from start of meal until glucose level changes, time that can be used for the insulin to diffuse to the blood, had the insulin been infused immediately with the meal. In SCLIDS the user can input a meal event to the system, however it is desirable to use less user interventions in systems designed to treat chronic conditions, since over time, users makes mistakes or forget to inform the system of such an event.

It is therefore desirable to provide a reliable simple system and method that can detect meals automatically, preferably without user intervention, that are not based on solely based on glucose measurements. Information regarding the onset of a meal from such system or such method may be used together with other systems and methods, to deliver drugs that need to be delivered in relation to the meal. Optionally such meal detection systems may indicate to a patients, or to provide signal to patients about meals that were taken without delivering drugs by injection or infusion systems, or without the patient taking the appropriate drug.

Particularly for diabetic patients it is desirable to have such an automatic meal detection system and method working with state of the art drug management, drug delivery system, SCLIDS or FCLIDS. Optionally such system and method should may further comprise an algorithms that preferably minimize the risk of false or missed drug infusion or indication to uptake.

In some embodiments of the present application the detection of a meal event may trigger an initial drug infusion to preempt and/or face expected post meal glucose increase. Some SCLIDS or FCLIDS systems decide on meal events based on several glucose readings. A better way is to have other methods that can detect meals since it take time from start of meal until glucose level changes, time that can be used for the insulin to diffuse to the blood, had the insulin been infused immediately with the meal. In SCLIDS the user can input a meal event to the system, however it is desirable to use less user interventions in systems designed to treat chronic conditions, since over time, users makes mistakes or forget to inform the system of such an event.

An optional embodiment of the present invention provides for a device for non invasive automatic meal detection that may be provided as a stand alone device. Optionally it may be an adjunct to a drug infusion pump and/or adjunct to an infusion set and/or embedded into the drug infusion pump or infusion set mechanism.

Optionally the device may also be part of and/or an adjunct to a drug patch placed on the skin or it may also be adjunct or part of a sensor intended to measure additional tissue parameters, for example an independent glucose sensor. Optionally the device for automatic meal detection may be invasive or implantable or non-invasive, optionally it may also have certain members that are invasive and/or implantable while other members are non invasive.

Optionally the device for automatic meal detection may further interface and/or work together with additional auxiliary device and/or methods provided to improve the pharmacokinetics and pharmacodynamics of the infused drug, preferably to enhance the drug absorption to the blood and reduce the time it takes for the drug to have effect on the target blood parameters, time lag between administration and effect.

Optionally the meal detection device for automatic meal detection may be used alone or in combination with other devices in a SCLDDS or FCLDDS.

An optional embodiment of the present invention provides a device for non invasive automatic meal detection adept at generating and providing a “meal signal” that may be used as input for an auxiliary device for example in the form of a medical device that may be used to deliver a treatment, medicament and/or drug to user such that the delivered treatment, medicament, and/or drug is provided relative to meals, or as reminder indication to patient to take drug.

Optionally the generated “meal signal” may be used in a SCLIDS or FCLIDS to initiate and/or start the delivery of “first phase insulin”. Optionally and preferably following the first phase insulin delivery a SCLIDS or FCLIDS may deliver additional amounts of insulin based on additional measurements, that may be independent of the meal signal, for example including but not limited to a blood glucose measurement.

Optionally the produced “meal signal” may also be used to activate a device that will enhance the drug absorption to the blood, for example, to enhance the blood perfusion in the tissue area where the drug is infused. Optionally the activation of an optional “blood perfusion enhancing” element with the detection of a meal may be necessary to achieve improved blood perfusion that will promote the delivery of the infused drug into the blood stream. Optionally initiating a blood perfusion elements prior to drug delivery, for example in the form of a “first phase” insulin may provide for infusing insulin faster to the blood, due to the treatment element since the capillaries are preferably in an “open” state when the drug is infused.

An optional embodiment of the present invention provides for the optional use of the “meal signal” to be used in a SCLDDS or FCLDDS to preferably trigger the onset of the delivery of at least one drug and/or medicament intended to treat obesity.

Optionally the meal signal according to the present invention may provide for delivering at least one or more treatment, drug and/or medicament toward a plurality of medical conditions, both acute and/or chronic. For example the system may be utilized to treat two chronic conditions simultaneously, for example diabetes and obesity, where the meal signal is utilized to initiate, prime and/or control the delivery of a first medicament treating obesity and optionally a second medicament to treat the diabetes. Optionally treatment may be geared toward only one of the two conditions being managed or toward both, or each in turn, or provided control by delivering a medicament in any controllable and safe combination.

Optionally and the system and method of the present invention may provide for controlling the drug delivery profile of any chronic or acute conditions, having varying affects and in some manner associated with the meal event. For example treatments, drugs and or medicaments having varying affect may be utilized to reduce blood glucose levels, reduce the amount of food and liquids absorbed in intestines, encourage the subject to consume smaller amount of food.

Optionally the “meal signal” may be used in a SCLIDS or FCLIDS optionally to first activate a device that will optionally and preferably enhance the blood perfusion at the site of a continuous glucose sensor and improve the sensor accuracy, optionally and preferably after obtaining a glucose reading from an optional sensor associated with the system.

Optionally a method according to the present invention may signal to the user a reminder about possible situation where insulin drug should be taken in case of a SCLIDS, or in case of a FCLIDS start the delivery of “first phase insulin”, in both cases, with or without activation of a device that will enhance the drug absorption to the blood. Optionally a second phase the SCLIDS or FCLIDS may deliver additional amounts of insulin based on additional measurements, for example blood glucose.

An optional embodiment of the present invention provides for generating the “meal signal” by a device that detects stomach volume change from its fasting volume, for example about 50 ml, to a volume that corresponds to food or drink uptake, for example up to 1 L and may expand in some cases even to 4 L. Optionally a meal signal detection device may analyze a volume rate of change the device to specifically indicate eating state from drinking state. For example, when drinking the volume rate of change is much larger than in case of eating. Optionally a volume change sensor may be realized by those skilled in the art from by a variety of transponders either acoustic or electronic or optic which send a signal and measure the properties of a returning signal to analyze changes in the tissue underneath or in the pathway of the transmitted signals.

An optional embodiment of the present invention provides for generating the “meal signal” by an optional device that is adept at detecting stomach sounds and/or activity which includes transducers positioned on the skin. Optionally the meal detection device may also include transducers that detect eating sounds and/or environmental sounds. Most preferably the device analyzes the different signals and sound sources and provides a “meal signal”.

An optional embodiment of the present invention provides for generating a “meal signal” with an optional device that detects electrical signals generated by stomach activity, for example when the stomach is digesting the food together. Optionally the such meal detection may be provided by transducers positioned on the skin to detect stomach sounds and other transducers that detect eating sounds and/or environmental sounds. Most preferably the device analyzes the different signals and sound sources and provides a “meal signal”.

An optional embodiment of the present invention provides for generating a “meal signal” with an optional device adept at detecting chewing or chewing and change in mouth glucose level. Optionally such a device may be provided in the form an implant in one or more tooth or “chewing signal”. Optionally a mouth glucose level may be utilized in combination with a “stomach signal” generated by any of the previously described devices. Optionally mouth glucose level as well as blood glucose level may be sensed by a glucose sensor implanted in one or more tooth. Most preferably the device analyses the various signals to derive an accurate “meal” and “meal content” signal. Optionally the size of such chewing detection device implanted in a tooth may be typically 1×1×1 mm or preferably 2×1×0.5 mm.

Optionally the chewing meal detection device may optionally include a first sensing area which detects pressure change to from chewing. Optionally the chewing meal detection device may optionally comprise a sensing area for an analyte level, for example glucose measured from within the mouth. Optionally, the chewing meal detection device may comprise a second sensing area for detecting an analyte level, for example including but not limited glucose in the blood, for example from the gum. =Optionally the chewing device may further comprise another sensing area which detects heart beats. Optionally an implantable device comprises electronics, memory and, power source. It is understood to those skilled that the optional memory element may be used to store data generated by the internal sensors of the device but it may optionally be used without the sensors as a memory element that may be read and from external device and optionally the data stored in the memory element may be modified by external device.

An optional embodiment of the present invention provides for a device adept at generating the “meal signal” by detecting chewing sounds and sounds generated by liquid and/or food passing through the throat. Optionally and preferably sensed data may then be analyzed and from it one may determine the amount of solid food and the amount of liquid.

Optionally meal event and it's “content” information may optionally and preferably be used by the system controller in conjunction with other measurements, for example including but not limited to glucose, may be used to control and/or adjust rate and mount of delivered insulin. For example, food intake which is mostly liquid will be absorbed faster from the intestine and may potently increase blood glucose faster. In the same way solid food which requires more chewing is expected to cause slower increase in blood glucose level and may be treated differently. Thus the information passed by the sensors to the system controller is used to fine tune the treatment by an algorithm that takes into account the different components of the meal.

An optional embodiment of the present invention may provide for estimating the volume of consumed foodstuff, and may then be utilized by system to determine the amount and rate of drug to be delivered. Optionally the volume of consumed food may be determined by a device that detects chewing or sounds from the throat.

An optional embodiment of the present invention the “meal signal” may be recorded together with additional physiological and activity parameters (i.e. temperature, blood glucose level, pulse rate, pedometer) for example by a controller of a SCLDDS or FCLDDS and preferably used to determine the amount of drug that such system should provide in future meal events, for example the next event of “meal”.

An optional embodiment of the present invention provides for a method for detecting a meal event of a user and generating a meal signal corresponding to the onset of a meal with an automatic meal detection device and communicating the meal signal to at least one or more auxiliary device for controlling the auxiliary device relative to and/or in response to the meal signal.

Optionally the at least one auxiliary device may be a drug delivery device and the control may optionally and preferably provided for priming the drug delivery device in response to the communicated meal signal.

Optionally the meal signal may detected with respect to a predefined threshold.

Optionally generating a meal signal further comprises evaluating and/or measuring the level of an analyte in response to the meal signal.

Optionally priming the drug delivery device comprises: Determining a drug dosage based on the meal signal; and Determining at least one or more delivery parameters of the drug relative to the meal signal.

Optionally priming the drug delivery device comprises: Determining a drug dosage based on the meal signal and the measured level of the analyte; and

Determining delivery parameters of the drug relative to the meal signal and the measured level of the metabolic analyte.

Optionally priming the delivery device includes control of a treatment element disposed in the drug delivery device aimed at accelerating the pharmacokinetics action of the drug.

Optionally the delivery parameters include the pharmacokinetic and pharmacodynamic properties of the drug.

Optionally the drug delivery device may for example including but is not limited to Semi Closed Loop Insulin Delivery Systems (SCLIDS), Fully Closed Loop Insulin Delivery Systems (FCLIDS), drug delivery device comprising a treatment element, therapeutic treatment device, Semi Closed Loop Drug Delivery Systems (SCLDDS), and Fully Closed Loop Drug Delivery Systems (FCLDDS).

Optionally the analyte may be glucose and the drug may be insulin.

Optionally the auxiliary device may be a gastric constriction ring or a gastric balloon.

Optionally the size of the gastric balloon may be adjusted and controlled according to the communicated meal signal

Optionally the size of the gastric constriction ring may be adjusted and controlled by way of increasing or decreasing its diameters according to the communicated meal signal.

Optionally the meal detection may provided with at least one or more sensors for example including but are not limited to volumetric sensor, Gastrointestinal sounds, oral sounds, or abdominal sounds; Gastrointestinal surface electromyogram (EMG), blood glucose sensor, saliva glucose sensor, interstitial fluid glucose sensor.

Optionally the at least one auxiliary device may be a device capable of communicating information to the user to enable/trigger user action or supply information to the user.

An optional embodiment of the present invention provides a system for detecting and responding to a meal event of a user comprising: a device for detecting meal event and generating a meal signal corresponding to the onset of a meal; and wherein the device interfaces and associates with at least one auxiliary device for controlling a response to the meal signal.

Optionally the auxiliary device may for example include but is not limited to an automatic drug delivery infusion set, drug delivery device, gastric ring, monitor, display, smart phone, call center, mobile telephone, gastrointestinal analyte measuring device, gastric balloon, gastric device, Semi Closed Loop Insulin Delivery Systems (SCLIDS), Fully Closed Loop Insulin Delivery Systems (FCLIDS), drug delivery device comprising a treatment element, therapeutic treatment device, Semi Closed Loop Drug Delivery Systems (SCLDDS), Fully Closed Loop Drug Delivery Systems (FCLDDS).

Optionally the auxiliary device triggers a response in relation to the meal signal.

Optionally the auxiliary device may be a drug delivery device comprising a treatment element to accelerate the delivery of a drug; wherein the auxiliary device may be further characterized in that the treatment element may be controlled in response to the communicated meal signal.

Optionally the treatment element may be activated in response to the meal signal to improve the pharmacokinetic and pharmacodynamic properties of the drug.

Optionally the drug is insulin.

Optionally the auxiliary device further comprises a display and wherein the auxiliary device may for example include but is not limited to a patient monitor, display, smart phone, a hand held device, call center, mobile telephone and wherein the display may be activated in response to the meal signal.

Optionally the activated display provides a user with a personal reminder for undertaking an activity for example including but are not limited to administrating a drug, undertaking exercise, reminder regarding eating habits.

Optionally the system may further comprise a master control device for interfacing with both the meal detection device and the at least one auxiliary device; and wherein the master control device may be adept at receiving the meal signal from the meal detection device and relaying and/or transmitting and/or converting the meal signal to a signal functional with the at least one auxiliary device.

Optionally the system may further comprise a secondary auxiliary device for example including but are not limited to a continuous analyte monitor, exercise device, pedometer, treadmill, VO2 treadmill, or a similar device adept for communicating at least one or more physiological parameters associated with the user, and wherein the first auxiliary device or the meal detection device may be provided with control of the system.

Optionally the system may further comprise a secondary auxiliary device for example including but are not limited to a continuous analyte monitor, exercise device, pedometer, treadmill, VO2 or a similar device adept for communicating at least one or more physiological parameters associated with the user, and wherein the master controller device interfaces with and controls the system.

Optionally the physiological parameters may for example including but are not limited to physical activity, temperature, blood glucose level, pulse, blood pressure, heart rate variability, pedometer, or a combination thereof, or the like.

It should be noted that the SCLIDS or FCLIDS serve only as a non limiting example of a drug that is delivered, and that there can be other drugs that can be used with Semi Closed loop Drug Delivery Systems (SCLDDS) or Fully closed Loop Drug Delivery Systems (FCLDDS), where the drug is automatically or manually infused to the SC tissue. It should also be noted that any of the SCLDDS or FCLDDS can deliver more than one drug to the tissue from one or more infusion pumps included in the system. Such SCLDDS or FCLDDS systems can deliver one or more drugs at various times, drugs such as insulin, glucagon, any of counter obesity drugs or any other drug that need to be delivered with respect to meals. It should also be noted that any of the SCLDDS or FCLDDS can include a drug delivery device which has an infusion set tube and a catheter attached to the tissue or the drug delivery device can be tubeless like a patch pump or a drug patch placed on the tissue having means to penetrate the skin and infuse the drug to the tissue or an implanted drug delivery device.

Although particular embodiments have been disclosed herein in detail, this has been done by way of example and for purposes of illustration only, and is not intended to be limiting. In particular, it is contemplated by the inventors that various substitutions, alterations, and modifications may be made without departing from the spirit and scope of the invention. Other aspects, advantages, and modifications are considered to be within the scope of the invention. The claims presented hereafter are merely representative of some of the embodiments of the invention disclosed herein. Other, presently unclaimed embodiments are also contemplated. The inventors reserve the right to pursue such embodiments in later claims and/or later applications claiming common priority.

Unless otherwise defined the various embodiment of the present invention may be provided to an end user in a plurality of formats, platforms, and may be outputted to at least one of a computer readable memory, a computer display device, a printout, a computer on a network or a user.

Unless otherwise defined, all technical and scientific terms used herein have the same meaning as commonly understood by one of ordinary skill in the art to which this invention belongs. The materials, methods, and examples provided herein are illustrative only and not intended to be limiting. Implementation of the method and system of the present invention involves performing or completing certain selected tasks or steps manually, automatically, or a combination thereof. Moreover, according to actual instrumentation and equipment of preferred embodiments of the method and system of the present invention, several selected steps could be implemented by hardware or by software on any operating system of any firmware or a combination thereof.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in order to provide what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1A-C are schematic block diagram of an exemplary device according to optional embodiments of the present invention;

FIG. 2A-D are schematic illustrative diagrams an exemplary device according to an optional embodiments of the present invention associated with the oral cavity;

FIG. 3A-C are schematic block diagram of an exemplary system according to optional embodiments of the present invention;

FIG. 4 is a flowchart depicting an exemplary method according to the present invention;

FIG. 5A-C are schematic diagrams of an optional system according to optional embodiments of the present invention.

### DESCRIPTION OF THE PREFERRED EMBODIMENTS

The principles and operation of the present invention may be better understood with reference to the drawings and the accompanying description. The following reference labels listed below are used throughout the drawings to refer to objects having similar function, meaning, role, or objective.

100 Meal detection device;

100a, b,c implanted device;

102 Sensor array module;

102a first Sensor array module;

102b second Sensor array module;

104 power source module;

106 communication module;

108 processor and/or controller module;

110 memory;

112 First member housing;

114 second member housing;

122 pressure sensor;

124 oral analyte sensor;

126 glucose sensor;

128 gyroscope sensor;

130 volumetric sensor;

132 accelerometer sensor;

134 inertial sensor;

150 auxiliary device

152 drug delivery system;

154 external monitor;

156 GI analyte measuring device;

158 gastric ring;

160 continuous glucose monitor

200 Oral Cavity;

202 tooth

300 meal detection system

310 main controller and interface;

501 integrated meal detection and drug delivery system

502 integrated meal detection and drug delivery system

503 integrated meal detection and drug delivery system

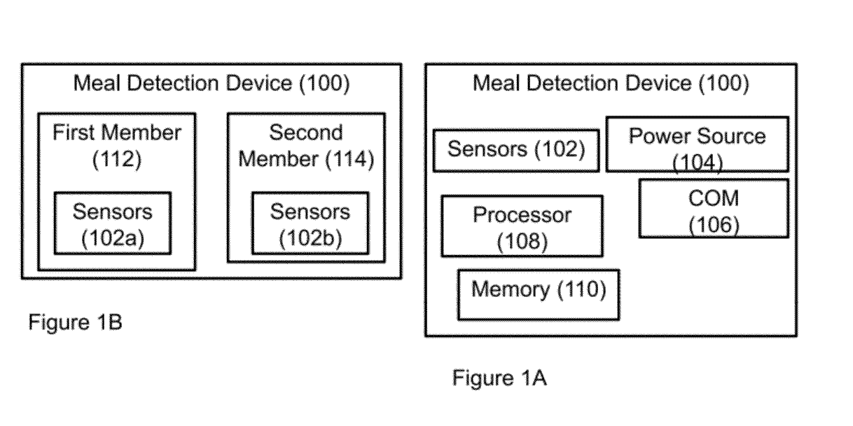

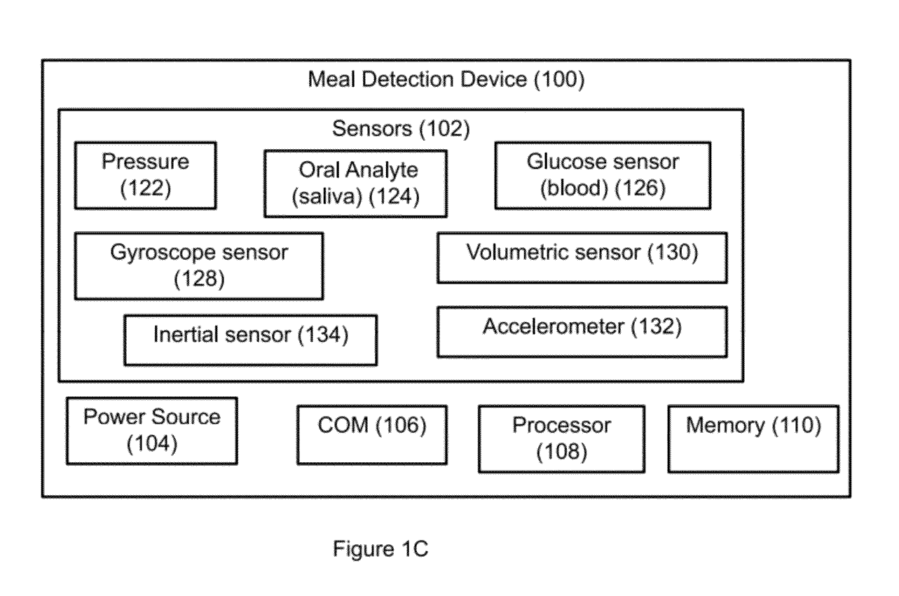

Referring now to the drawings, FIGS. 1A-C show schematic block diagrams of an exemplary device according to the present invention for detecting a user's meal state.

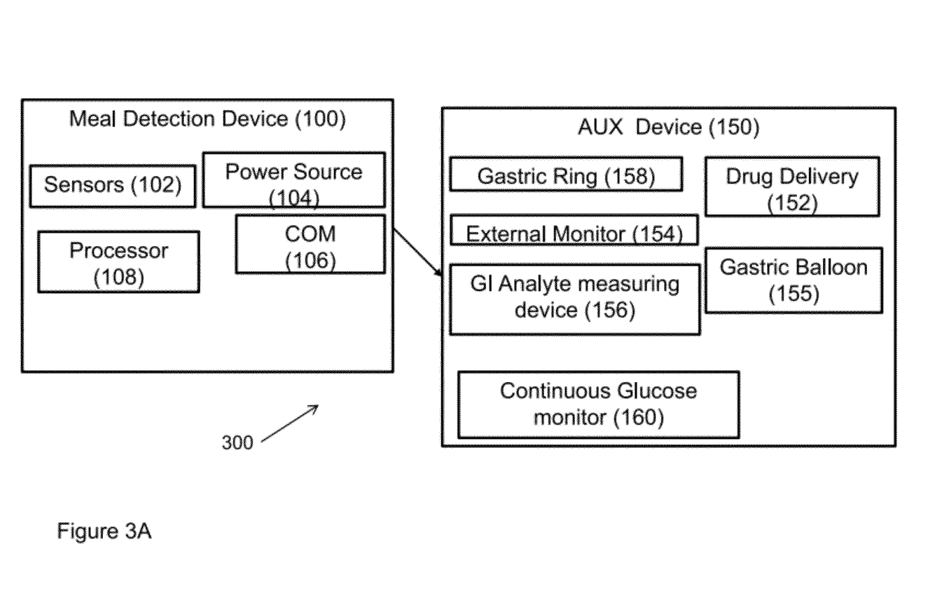

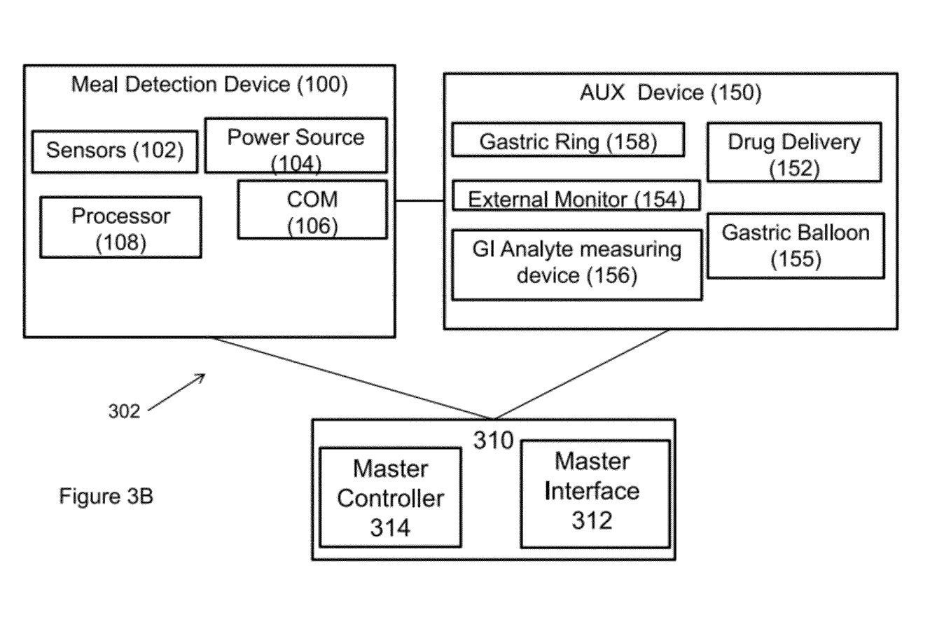

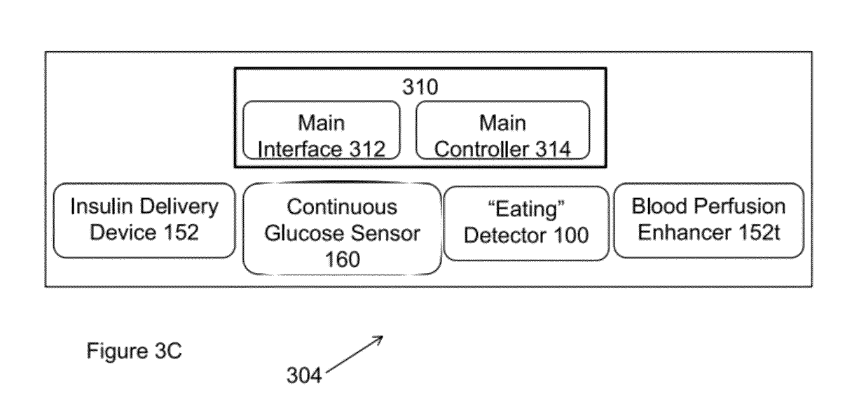

FIG. 1A depicts meal detection device 100 comprising a sensor module 102, a power source 104, a communication module 106, memory 110 and a processor 108. Optionally and preferably sensor module 102 comprising at least two or more sensors adept for determining the meal state of a user by sensing when a user is eating. Most preferably when such a meal state is determined by processor 108 and communication module provide for generating and communicating a meal signal to auxiliary devices as will be described in FIG. 3. Optionally and preferably the meal state and associated sensor results are stored in memory 110 providing for historical comparison, and analysis in controlling and determining the overall health state of a user.

Optionally device 100 may be realized as a device provided in a single housing, most preferably such housing is provided form biocompatible materials. Optionally device 100 may be realized in a plurality of optional orally implantable forms for example including but not limited to a crown, retainer, dentures, dental implant, intra tooth device, dental surface, intrapulpal device, intradental device, mucosal implant, sublingual implant, gingivae implant, inferior frenulum implant, superior frenulum implant, lingual frenulum implant, supra gingivae implant, sub-gingivae implant, intra-gingivae implant, or the like.

Optionally meal detection device may be realized as a device that is coupled to or otherwise attached to the skin of a user, for example over the throat, face, head, behind the ear, chest or abdominal surface. Optionally, displacement of device 100 over the skin may provide for detection electromyography signals, gastric sounds, electro cardiac pulses, or volumetric measurements of the gastrointestinal system.

Optionally as shown in FIG. 1B device 100 may be realized in a two part housing comprising a first member 112 and a second member 114. Optionally each first and second member may optionally comprise at least one sensor. Most preferably at least one of the first member 112 or second member 114 comprises at least one and more preferably at least two sensors. Optionally first member 112 and second member 114 may interact in a master and slave configuration.

FIG. 1C provides a closer depiction of the optional sensors comprising sensor module 102. Optionally device 100 may comprise at least one and more preferably at least two or more sensor for example including but not limited to pressure sensor 122, oral analyte sensor 124, glucose sensor 126, gyroscope sensor 128, volumetric sensor 130, accelerometer sensor 132, inertial sensor 134, any combination thereof or the like. Optionally and preferably each sensor type is adept at detecting a particular state that may be associated with a meal. For example, a volumetric sensor may sense the changing volume of the Gastrointestinal system to determine the meal state while differentiating between fluid intake and solid intake. For example a glucose monitor may identify the rate of change of oral glucose levels indicating a meal. For example, a salivary analyte sensor may sense the increase in salivary amylase in the saliva to infer a meal state. For example a gyroscopic sensor and/or an inertial sensor may sense the movement of the upper and lower jaw with respect to one another. For example a microphone may sense chewing sounds. For example a pressure sensor may detect pressure changes within the oral cavity, or along the surface of at least one tooth or at least two teeth.

Most preferably at least two or more sensors may be used to determine and/or cross reference the meal state of a user.

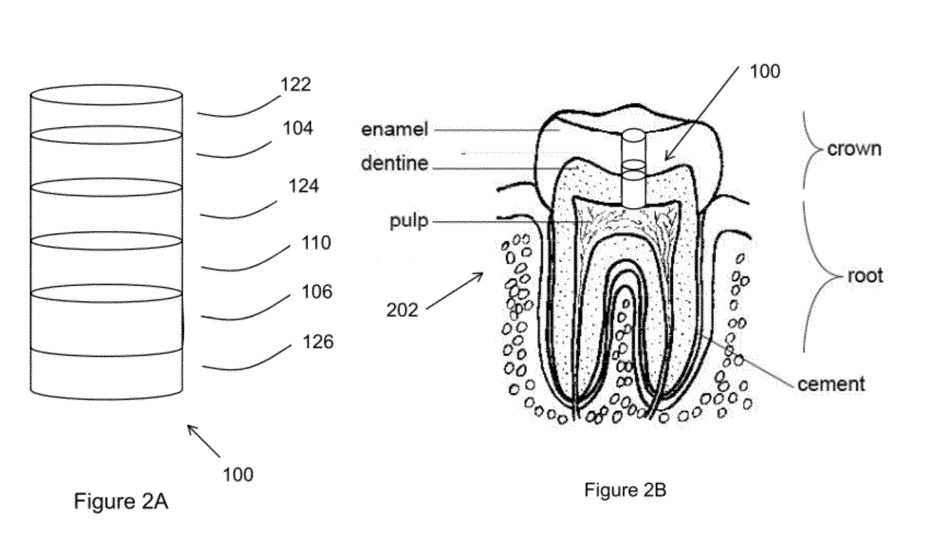

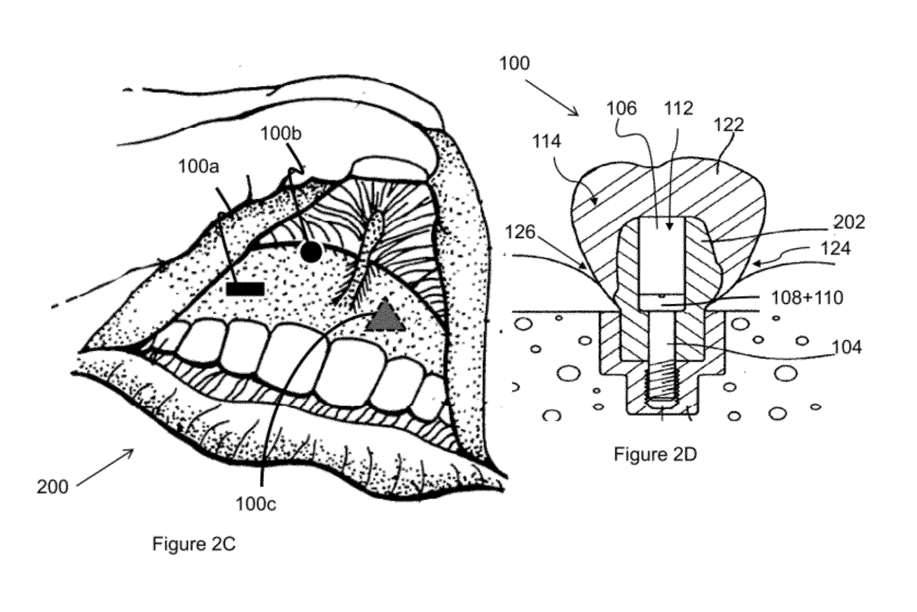

FIG. 2A depicts an optional non limiting illustrative example of the meal detection device 100, comprising at least two sensors in the form of a pressure sensor 122 and glucose sensor 126. Device 100 as shown in FIG. 2A, further comprises a biocompatible housing (not shown) most preferably to provide for packaging device 100 in accordance with the appropriate implanted location (FIG. 2C). Device 100 is provided with an energy source from power source module 104, for example in the form of a battery, capacitor or the like. Device 100 further comprises communication module 106 to provide for communicating a detected meal signal, for example as provided by pressure sensor 122 disposed about the external surface of device 100. Optionally communication module may be realized with a variety of optional wireless communication protocols for example optical, cellular, Bluetooth, RFID, or the like. Most preferably device 100 further comprises memory 110 for storing historical data associated with sensed meals, and analyte levels. Optionally memory 110 may be communicated and flushed via communication module 106. Optionally and preferably device 100 may be provided with at least one and more preferably at least two analyte sensor providing for sensing the state of an analyte associated with the meal state of a user. For example device 100 of FIG. 2A is provided with first sensor in the form of an oral analyte sensor 124 preferably providing for sensing analyte from saliva and a second sensor 126 optionally in the form of a blood glucose sensor. Optionally analyte sensor 124 and glucose sensor 126 may be used in conjunction with pressure sensor 122 to determine the meal state of a user and to identify the level of an analyte for example glucose both from the saliva, as may be provided for with sensor 124 and from blood as may be provided by sensor 126. Optionally and preferably all three data forms may be combined to determine the meal signal communicated with communication module 106 which would be better reflect the meal state of a user so as to control other devices for example drug delivery device as will be described in FIG. 3-5. Therefore most preferably the meal signal comprises a plurality of information that may be associated directly with the meal state and at least one or more analyte for example glucose.

FIG. 2B illustrates non limiting example of a “meal detection” device 100 depicted in the form of an intrapulpal device that is implanted in the tooth, the image showing an implant 100 as shown in FIG. 2A, implantation within a tooth. FIG. 2B depicts the device described in 2A in its implanted form within a tooth 202, where optionally and preferably the onset of a meal may be detected with a sensor 122 disposed about the surface of the tooth and adapted to detecting chewing optionally by pressure changes about it surface. Optionally pressure sensor 122 may be provided from a plurality of materials for example biocompatible PVDF materials. A second sensor in the form of a glucose senor, optionally and preferably a glucose sensor 126 where blood or interstitial fluid is utilized as an analyte. Optionally sensor 126 may be disposed within the pulp of a tooth to allow measuring blood glucose levels where optionally and preferably the timing of activation of the glucose monitor is based on the detection of the meal, preferably provided by pressure sensor 122.

FIG. 2C provides a further optional depiction of implantation sites about the oral cavity 200. Optionally device 100a, b, c may be associated with and/or implanted within the oral cavity to provide for detecting the onset of a meal and generating a meal signal while measuring glucose levels. Optionally and preferably the implantation site about the oral cavity 200 may be chosen based on optimization patient comfort, quality of meal detection and meal signal, and the ability to detect an analyte for example glucose.

For example device 100a is depicted as a supra-gingivae implant about the gum surface where a blood glucose sensor 126 may utilize the blood source about the gum surface. Another optional implantation site about the oral cavity is shown with device 100b where it is implanted about the mucosa near the gum and lip border. Optionally as shown with device 100c the device may be implanted sub-gingivae below the gum line or palate, shown in broken line, to optimize both a subject's comfort and quality and availability of signals.

FIG. 2D provides an illustrative non limiting depiction of an optional embodiment of the present invention where meal detection device 100, as described in FIGS. 1A-C, is provided in the form of a crown. Crown 100 is preferably placed over a tooth base 202 within the oral cavity. Optionally crown 100 is provided from two housing first member housing 112 and second member housing 114. Optionally first member housing 112 comprises the electronics associated with meal detection device 100 comprising power source 104, communication module 106, processor 108, and memory 110. Optionally second member housing 114 comprises a plurality of sensors for depicting the meal state of a user. Optionally and preferably the crown surface comprises a pressure sensor 122 able to detect chewing and/or grinding of the crown 100. Optionally and preferably second housing member 114 may further comprise at least one or more analyte sensors as shown two analyte sensor are disposed about the lower surface of the crown most preferably disposed about the surface that is in contact with the gingivae surface. Optionally glucose sensor 126 and saliva analyte sensor 124 are disposed as shown.

FIGS. 3A-C depict a meal detection system according to optional embodiments of the present invention wherein system 300 comprises at least one a meal detection device 100 in communication with at least one or more auxiliary device 150. Optionally any number of auxiliary device may associate and or interface with meal detection device 100, for example including but not limited to gastric ring 158, external monitor 154, gastrointestinal measuring device 156, gastric balloon 155 and drug delivery device 152, or the like.

FIG. 3A depict system 300 wherein meal detection device 100 and optional auxiliary devices communicate and interface directly with one another, optionally via communication module 106 disposed in meal detection device 100 and a processor intrinsic to the optional auxiliary device 150 being used. For example, a meal detection signal produced with device 100 may cause a gastric ring 158 implanted in a user to either clamp or release based on the sensed meal state of the user. For example, a gastric balloon may inflate or deflate relative to the meal signal communicated with device 100.

FIG. 3B-C provide an exemplary block diagram to illustration an optional embodiment of the present invention for a system 302, 304 comprising an optional auxiliary device 152 and a meal detection device 100 that work together to better control of the delivery of the drug with respect at least one or more parameters associated with the meal state, where the interaction between them is interfaced with a master controller device 310. Master controller device 310 provides a transitional device that is capable of communication and controlling both meal detection device 100 and optional auxiliary devices 150.

FIG. 3C provides a specific depiction of FIG. 3B in system 304 where a meal detection device 100 is interfaced by master controller 310 with a plurality of drug delivery devices specific to insulin, for example insulin delivery device 152, continuously glucose sensor 160, and a blood perfusion enhancer 152t that is associated with delivery device 152.

Most preferably a meal detection device 100 and a drug delivery system interface with one another via a master controller 310 such that the detected meal and generated meal signal with an optional device 100 may be communicated to, for example with communication module 106, and received by the insulin delivery system 152 to control the at least one or more parameters associated with the drug delivery for example including but not limited timing of delivery, dosage of delivery, rate of delivery or the like drug delivery parameters. Optionally insulin delivery system 152 may further process the meal signal received from meal detecting device 100 via interface master controller 310 to activate a blood perfusion enhancer for improving the insulin delivery time and insulin pharmacokinetic and pharmacodynamic properties and based on the measured blood glucose level determined with continuous glucose sensor 160.

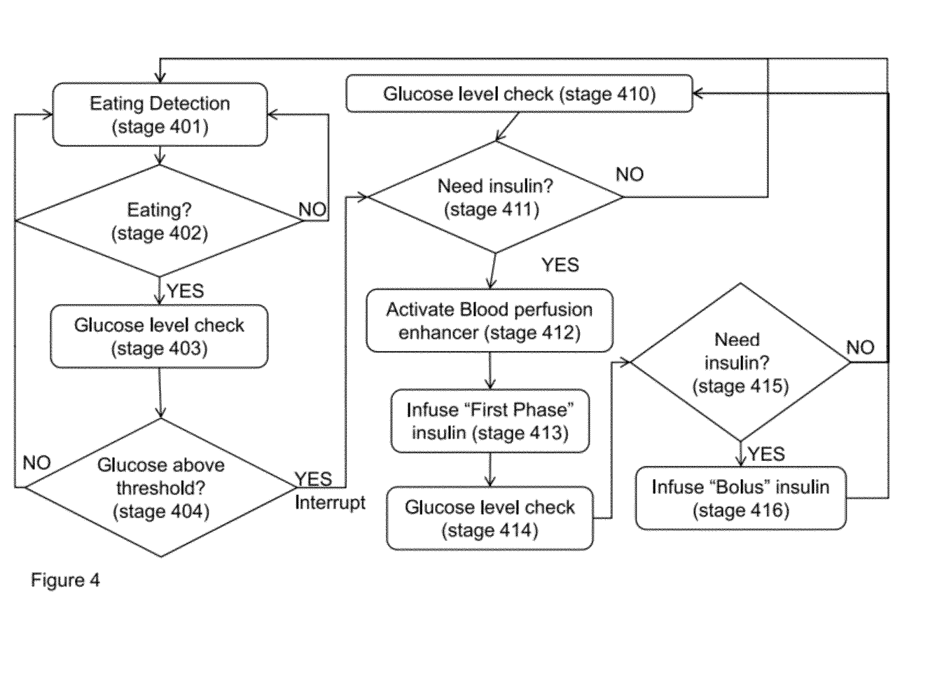

FIG. 4 provides a flowchart depicting the method of integrated control of meal detection device 100 providing a meal signal and a drug delivery system for the delivery of insulin, specifically a closed loop insulin delivery system is depicted. It will be appreciated by one skilled in the art that although embodiments of the present application are described with respect to control of diabetes and the administration of insulin, the invention and it embodiments should not be understood or construed as limited to an insulin delivery system. In particular the same embodiments could just the same be applied to any analyte and drug delivery system that relate to caloric or food intake, for example the control of a drug in response to levels of detectable blood fats such as cholesterol or triglycerides when a meal is detected.

In stage 401, food is continuously detected for example with device 100 using at least one or more preferably a plurality of sensors to detect a user's meal state. Next in stage 402 a detected event is questioned to detect if it is a true meal event rather than a false alarm meal. For example a true meal state would be detected if a person was to ingest solids and liquids or based on a given volume consumed. While a non meal state, may for example be the detection of intake of water or a simple conversation or sleep state where a an individual grinds their teeth but without any caloric intake.

Next in stage 403 once an meal state has been established, optionally and most preferably a meal signal is generated and optionally communicated with device 100 via communication module 106. Most preferably a glucose level check is performed, optionally by an intrinsic glucose sensor 126 to device 100 or via an external sensor optionally provided in the form of an independent continuous glucose sensor 160 or a glucose sensor 152g as part of drug delivery system 152. Most preferably if external glucose sensor glucose sensor 160 and/or glucose sensor 152g are implemented the meal signal is communicated via communication module 106 to a main interface controller 310.

Next in stage 404 glucose level is evaluated relative to a threshold. If a threshold is not crossed eating detection is continuously monitored. If a threshold is crossed the meal signal is preferably communicated comprising data indicative of the glucose state and the meal state to a glucose drug delivery system 152, for further control and action for example delivery of insulin to control and/or maintain blood glucose homeostasis or balance. Optionally and preferably communication is facilitated with device 100 via 1 communication module 106 and transmitted to interface controller 310 for relaying to drug delivery system 152.

Next in stage 411 drug delivery system 152 evaluates the glucose levels communicated form device 100 via interface controller 310 and optionally further weighs its internal glucose level check provide in stage 410, to determine if action, optionally in the form of insulin delivery is required. If it is determined that no action is required drug delivery system 152 continues to monitor glucose levels via stage 410. If action is required a plurality of optional activities may be undertaken intrinsically by drug delivery device 152. For example, in stage 412 through to stage 416 the drug delivery device 152 performs its intrinsic test and measures of glucose to delivery the appropriate, safe amount of insulin to maintain the homeostatic state of blood glucose level in the user, optionally the measures taken to maintain the blood glucose level depend on the intrinsic tools available drug delivery system at hand. For example, as shown in stage 412, some system may comprise a blood perfusion enhancing mechanism to improve the pharmacokinetic and pharmacodynamic properties of the delivered insulin. Other systems not comprising such tools will optionally delivery insulin and continuously and interactively check the glucose levels as shown in stages 413-416 until the glucose levels have reached homeostatic or a similar stable state.

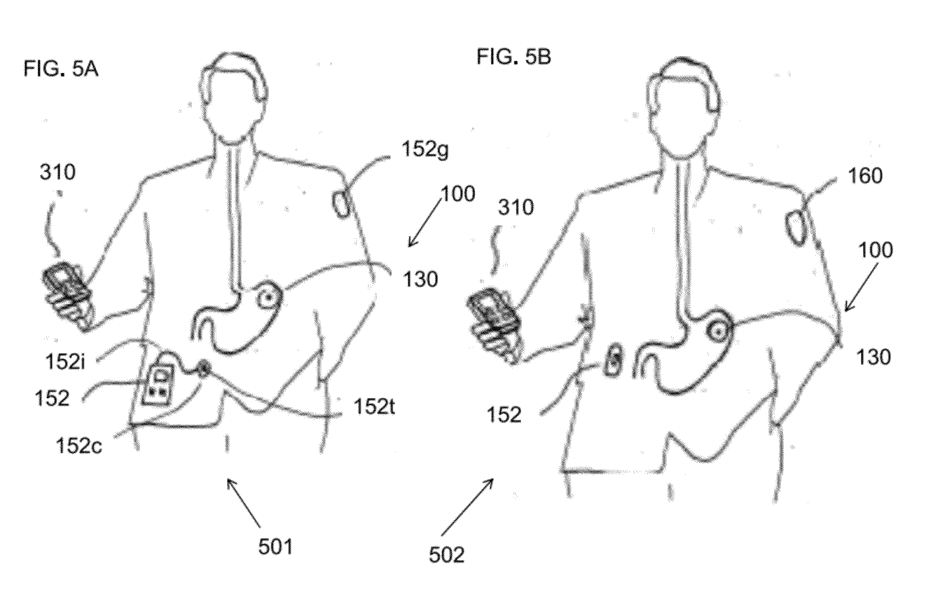

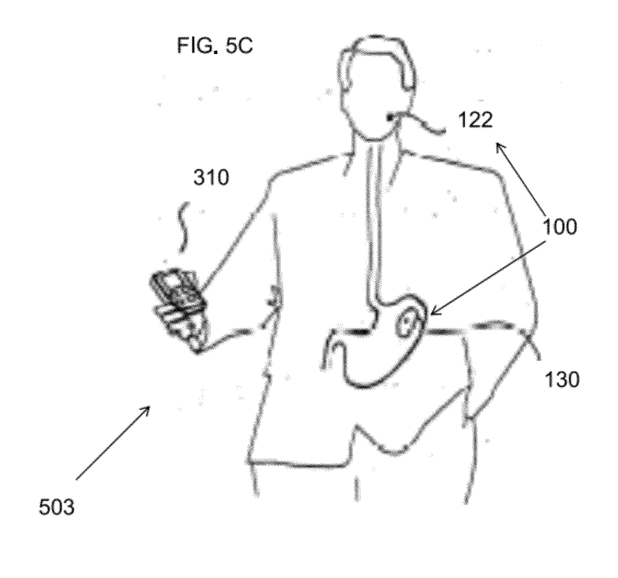

FIG. 5A-C provide an illustration of optional embodiment of the present invention for a system comprising a drug delivery device and a meal detection device 100 that work together to better control of the delivery of the drug with respect at least one or more parameters associated with the drug. Most preferably a meal detection device 100 and a drug delivery system interface with one another via a master controller 310 such that the detected meal and generated meal signal with an optional device 100 may be communicated to, for example with communication module 106, and received by the drug delivery system to control the at least one or more parameters associated with the drug delivery for example including timing of delivery, dosage of delivery, rate of delivery or the like drug delivery parameters.

FIG. 5A depicts system 501 comprising, a controller interface device 310 for interfacing between a drug delivery device 152 and meal detection device 100.

Optionally drug delivery device 152 comprising an infusion set 152i, a catheter 152c, a blood perfusion enhancer 152t and a continuous glucose sensor 152g. Optionally meal detection device 100 comprises at least two volumetric sensor 130 for sensing the changing stomach volume providing for meal detection and generation of meal signal.

Optionally and preferably drug delivery device 152 and meal detection device 100 may individually function independently of one another where a shared controller 310 provides for interfaced both delivery device 152 and meal detection 100 such that the meal signal can control and positively affect the drug delivery parameters of device 152.

Optionally continuous glucose monitor 152g, presented here as part of drug delivery system 152 may be provided as a third auxiliary device that is collectively controlled with controller 310, as shown in FIG. 5B depicting system 502, wherein continuous glucose monitor 152g is realized as an independent device. Optionally controller 310 may provide for integrating data from three or more data points and/or sources to bring about stable and proper control of a drug delivery system 152, for example in the form of insulin, as shown here, that depends on a delicate balance between a plurality of factors for example the timing of a meal, via meal detection device 100 and, current blood glucose levels, provided by continuous glucose monitor 160 providing overall control and timing of the delivery of insulin in order to sustain, and maintain a controllable, stable and safe blood glucose level.

FIG. 5C depicts system 503 according to an optional embodiment where meal signal device 100 is realized with chewing sensor 122 for example provided in the form of a pressure sensor comprising a biocompatible PVDF. Optionally PVDF layer may be placed about the outer surface of at least one crown, or optionally two crowns, that are securely associated and/or placed over at least one tooth 202 or two teeth that correspond with one another while chewing food within the oral cavity 200. The interface controller 310 provides for dual control and interface between chewing sensor 122, and stomach volume sensor 130 and subcutaneous drug delivery device 152 (not shown) as previously described.

While the invention has been described with respect to a limited number of embodiment, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not described to limit the invention to the exact construction and operation shown and described and accordingly, all suitable modifications and equivalents may be resorted to, falling within the scope of the invention.

Having described a specific preferred embodiment of the invention with reference to the accompanying drawings, it will be appreciated that the present invention is not limited to that precise embodiment and that various changes and modifications can be effected therein by one of ordinary skill in the art without departing from the scope or spirit of the invention defined by the appended claims.

Further modifications of the invention will also occur to persons skilled in the art and all such are deemed to fall within the spirit and scope of the invention as defined by the appended claims.

While the invention has been described with respect to a limited number of embodiments, it will be appreciated that many variations, modifications and other applications of the invention may be made.

1-46. (canceled)

47. A system for detecting and responding to a meal event of a user comprising:
a. A device for detecting the meal event and generating a meal signal corresponding to the onset of a meal, the device configured to detect at least one or more signal indicative of said meal event selected from the group consisting of: a gastrointestinal volume change, acoustic signals originating from the user's stomach, acoustic signals originating from the user's throat, chewing, oral sounds, oral analyte, any combination thereof; and
b. wherein said device interfaces and associates with at least one auxiliary device for controlling a response to said meal signal.

48. The system of claim 47 wherein said device for detecting a meal event is selected from the group consisting of:
i. a device configured to detect a gastrointestinal volume change;
ii. a device configured to detect acoustic signals originating from at least one of the user's stomach or throat;
iii. a device configured to detect a pressure differential within a user's mouth;
iv. a device configured to detect acoustic signals originating from at least one transducer positioned on a surface of the user's skin;
v. a pressure sensor and a glucose sensor;
vi. a device that uses data indicative of an analyte in a user's saliva, a user's blood glucose, and a pressure level in a user's mouth;
vii. a device that uses data indicative of a gastrointestinal volume change;
viii. a device implanted into a user's tooth.

49. The system of claim 48 wherein said meal event detection is provided by at least two devices.

50. The system of claim 47 wherein said auxiliary device is selected from the group consisting of: an automatic drug delivery infusion set, drug delivery device, gastric ring, monitor, display, smart phone, call center, mobile telephone, a hand held device, gastrointestinal analyte measuring device, gastric balloon, gastric device, Semi Closed Loop Insulin Delivery Systems (SCLIDS), Fully Closed Loop Insulin Delivery Systems (SCLIDS), drug delivery device comprising a treatment element, therapeutic treatment device, Semi Closed Loop Drug Delivery Systems (SCLDDS), Fully Closed Loop Drug Delivery Systems (FCLDDS), device that provides treatment drugs or medicament to reduce blood glucose levels, reduce the amount of food and liquids absorbed in intestines, device to encourage the subject to consume smaller amount of food, device to enhance drug absorption to the blood, device to enhance blood perfusion in the tissue area where drug is infused, continuous glucose sensor, device to enhance blood perfusion in the tissue area where a glucose sensor is positioned, device that provides a reminder to the user to take medicament.

51. The system of claim 50 wherein said auxiliary device triggers a response in relation to said meal signal.

52. The system of claim 51 wherein said auxiliary device is a drug delivery device comprising a treatment element to accelerate the delivery of a drug; wherein said auxiliary device is further characterized in that said treatment element is controlled in response to said communicated meal signal.

53. The system of claim 51 wherein said treatment element is activated in response to said meal signal to improve the pharmacokinetic and pharmacodynamic properties of said drug.

54. The system of claim 53 wherein said drug is insulin.

55. The system of claim 51 wherein the response in relation to said meal signal is to provide a user with a personal reminder for undertaking an activity selected from the group consisting of administrating a drug, undertaking exercise, or providing a reminder regarding eating habits.

56. The system of claim 47 further comprising a master control device for interfacing with both said meal detection device and said at least one auxiliary device; and wherein said master control device is configured to receive said meal signal from said meal detection device and relay, transmit, and/or convert said meal signal to a signal functional with said at least one auxiliary device.

57. The system of claim 47 wherein said device to detect acoustic signals originating from the user's stomach and/or gastrointestinal volume change is at least one microphone.

58. The system of claim 47 wherein said meal detection device is composed of a two part housing having a first member and a second member.

59. The system of claim 58 further comprising auxiliary device that delivers medicament to the user.

60. A method for detecting a meal event of a user with the system of claim 50, the method including generating a meal signal corresponding to the onset of a meal with said automatic meal detection device configured to detect at least one or more signal indicative of said meal event selected from the group consisting of: a gastrointestinal volume change, acoustic signals originating from the user's stomach, acoustic signals originating from the user's throat, chewing, oral sounds, oral analyte, any combination thereof; and communicating said meal signal to at least one or more auxiliary device for controlling said auxiliary device relative to or in response to said meal signal.

61. The method of claim 60 wherein said at least one auxiliary device is a drug delivery device and wherein said control provides for priming said drug delivery device in response to said communicated meal signal; wherein said priming comprises at least one selected from the group consisting of:
a. accelerating the pharmacokinetics action of the drug;
b. determining a drug dosage based on the meal signal; and determining at least one or more delivery parameters of the drug relative to the meal signal comprising the pharmacokinetic and pharmacodynamic properties of said drug;
c. Determining a drug dosage based on the meal signal and the measured level of an analyte.

62. The method of claim 60 wherein said meal signal is detected with respect to a predefined threshold.

63. The method of claim 60 wherein generating a meal signal further comprises evaluating/measuring the level of an analyte in response to said meal signal.

64. The method of claim 63 wherein said analyte is glucose and wherein said drug is insulin.

65. The method of claim 60 wherein said response reduce the amount of food and liquids absorbed in intestines, and/or encourage the subject to consume smaller amount of food
